(19) United States
(12) Patent Application Publication
(10) Pub. No.: US 2010/0168422 A1
(43) Pub. Date: Jul. 1, 2010

(54) METHODS AND INTERMEDIATES USEFUL IN THE SYNTHESIS OF HEXAHYDROFURO [2,3-B]FURAN-3-OL

(76) Inventor: Weiping Chen, Liverpool (GB)

Correspondence Address: FISH & RICHARDSON P.C., P.O. BOX 1022, MINNEAPOLIS, MN, 55440-1022, US

(21) Appl. No.: 12/345,838

(22) Filed: Dec. 30, 2008

(51) Int. Cl. C07D 413/14; C07D 493/04; C07D 307/32; C07D 265/30; C07C 291/04; C07D 417/14; C07F 9/655

(52) U.S. Cl. 544/152; 549/464; 549/475; 544/170; 564/297; 548/203; 549/220; 548/242

(57) ABSTRACT

Provided herein are compounds and methods useful for preparing hexahydrofuro[2,3-b]furan-3-ol. Hexahydrofuro[2,3-b]furan-3-ol can be efficiently synthesized in four steps from readily available starting materials.

### TECHNICAL FIELD

The present disclosure relates to methods for the synthesis of hexahydro-furo[2,3-b]furan-3-ol compounds and novel synthetic intermediates useful therein. Optically enriched hexahydro-furo[2,3-b]furan-3-ol, e.g., the 3R, 3aS, 6aR isomer, can be prepared using the methods described herein.

### BACKGROUND

The compound (3R,3aS,6aR)-hexahydro-furo[2,3-b]furan-3-ol is a pharmacological moiety present in various protease inhibitors that have proven useful in the treatment of human immunodeficiency virus (HIV) and hepatitis C virus (HCV). Examples of such inhibitors include HIV protease inhibitors, darunavir, brecanavir, UIC-94003, and GS-9005. Inhibition of the protease enzymes has proven to be an effective treatment against AIDS, and can be used in combination with reverse transcriptase inhibitors. In view of the importance of the above protease inhibitors and the consequent need to manufacture these compounds there exists a need to develop cost effective and efficient methods of preparing hexahydro-furo[2,3-b]furan-3-ol compounds.

### SUMMARY

Described herein are novel methods for preparing hexahydro-furo[2,3-b]furan-3-ol and novel intermediates useful in the synthesis of the same. The synthetic routes employed herein can be used to prepare diastereomerically and/or enantiomerically enriched hexahydro-furo[2,3-b]furan-3-ol from inexpensive and readily available starting materials.

Certain embodiments relate to a process for preparing a compound of Formula 7:

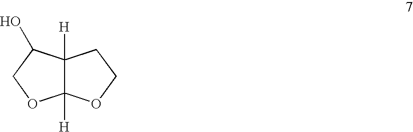

comprising
(a) combining a reducing agent and a compound of Formula 5:

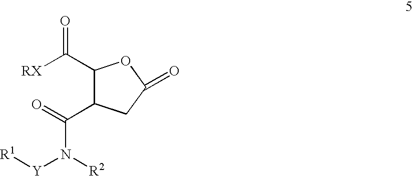

or a salt thereof, where X can be S or O; Y can be O, —N(R³)—, or a bond; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; R¹⁰ independently for each occurrence can be hydrogen, alkyl, alkenyl, alkynyl, cycloalkyl, heterocycloalkyl, aryl, aralkyl, heteroaryl, or heteroaralkyl; or R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together form can represent a 5-8 membered optionally substituted heterocyclic ring; and m is 0-8; to give a compound of Formula 6:

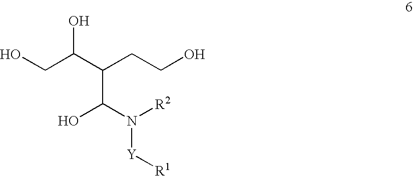

or a salt thereof, where each of Y, R¹ and R² is as defined above; and combining the compound of Formula 6 with an acid to give the compound of Formula 7.

Certain embodiments relate to the aforementioned process, where the reducing agent can be MBH₄, MHB(R³)₃, MH₂B(R⁴)₂, MH₃BR⁴, MHB(OR⁴)₃, MH₂B(OR⁴)₂, MH₃BOR⁴, MAlH₄, MHAl(OR⁴)₃, MH₂Al(OR⁴)₂, MH₃Al(OR⁴), HB(R⁴)₂, H₂BR⁴, BH₃, H₂Al(R⁴)₂, H₂AlR⁴, or H₃Al;
M is Li, Na, K, R³₄N, ½Zn or ½Ca; and R⁴ can be alkyl or aralkyl.

Certain embodiments relate to any one of the aforementioned processes, where the acid can be hydrochloric acid, hydrobromic acid sulfuric acid, phosphoric acid, nitric acid, metal hydrogen sulfate, metal dihydrogen phosphorate, trifluoroacetic acid, trichloroacetic acid, citric acid, oxalic acid, tartaric acid, oxalic acid, formic, methanesulfonic acid, trifluoromethanesulfonic acid, benzenesulfonic acid, p-toluenesulfonic acid, 10-camphorsulfonic acid, 1,5-napthalene disulfonic acid, or 1,2-ethane disulfonic acid.

Certain embodiments relate to any one of the aforementioned processes where, the compound of Formula 7 can be

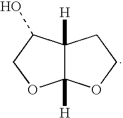

Certain embodiments relate to any one of the aforementioned processes, where R can be alkyl.

Certain embodiments relate to any one of the aforementioned processes, where Y can be a bond; and each of R¹ and R² independently for each occurrence can be alkyl, cycloalkyl, aryl, or —[C(R¹⁰)₂]ₘ—R¹¹, wherein independently for each occurrence R¹⁰ can be hydrogen or alkyl, R¹¹ is alkoxy; or R¹ and R² taken together with the nitrogen to which they are bonded can represent a 5-8 membered optionally substituted heterocyclic ring.

Certain embodiments relate to any one of the aforementioned processes, where Y is a bond; R is alkyl; and each of R¹ and R² independently for each occurrence is alkyl, cycloalkyl, aryl, or —[C(R¹⁰)₂]ₘ—R¹¹, wherein independently for each occurrence R¹⁰ is hydrogen or alkyl, R¹¹ is alkoxy; or R¹ and R² taken together with the nitrogen to which they are bonded represent a 5-8 membered optionally substituted heterocyclic ring.

Certain embodiments relate to any one of the aforementioned processes, where Y can be a bond; R can be methyl, ethyl, n-propyl, or i-propyl; X is O; and R¹ and R² taken together with the nitrogen to which they are bonded can form a heterocyclic ring selected from piperidine, pyrrolidine, or morpholine.

Certain embodiments relate to any one of the aforementioned processes, further comprising combining a compound of Formula 3:

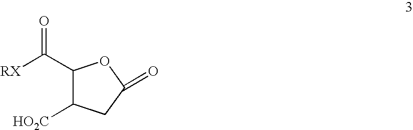

or a salt thereof, where X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m is 0-8; a coupling agent; and a compound of Formula 4:

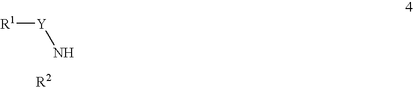

or a salt thereof, where Y can be O, —N(R³)—, or a bond; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; to give the compound of Formula 5.

Certain embodiments relate to the aforementioned process, where the coupling agent is dicyclohexylcarbodiimide (DCC), diisopropylcarbodiimide (DIC), N,N′-carbonyldiimidazole (CDI), 3-(diethoxyphosphoryloxy)-1,2,3-benzotriazin-4(3H)-one (DEPBT). 1-ethyl-3-(3-dimethyllaminopropyl)carbodiimide hydrochloride (EDCL), 2-(7-aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU). 2-(6-chloro-1H-benzotriazole-1-yl)-1,1,3,3-tetramethyluronium hexafluorophosphate (HCTU), benzotriazole-1-yl-oxy-tris-pyrrolidino-phosphonium hexafluorophosphate (PyBOP), bromo-tris-pyrrolidino-phosphonium hexafluorophosphate (PyBrOP), O-(7-azabenzotriazole-1-yl)-N,N,N′,N′-tetramethyluronium tetrafluoroborate (TATU), 2-(1H-benzotriazole-1-yl)-1,1 3,3-tetramethyluronium tetrafluoroborate (TBTU), N,N,N′,N′-tetramethyl-O-(3,4-dihydro-4-oxo-1,2,3-benzotriazin-3-yl)uronium tetrafluoroborate(TBTU) O-(N-succinimidyl)-1,1,3,3-tetramethyluronium tetrafluoroborate (TSTU). 4-(4,6-dimethoxy-1,3,5-triazin-2-yl)-4-methylmorpholinium chloride (DMTMM), 2-chloro-4,6-dimethoxy-1,3,5-triazine/N-methylmorpholine (CDMT/NMM), oxalyl chloride, SOCl₂, SO₂Cl₂, POCl₃, PCl₃, PCl₅, PBr₃, PBr₅, POBr₃, pivaloyl chloride, or pivaloyl anhydride.

Certain embodiments relate to any one of the aforementioned processes, where the coupling agent can be CDI, DMTMM, or EDCL, and the compound of Formula 4 can be morpholine.

Certain embodiments relate to any one of the aforementioned processes, where R can be methyl, ethyl, n-propyl, or i-propyl; X can be O; and the compound of Formula 4 can be morpholine.

Certain embodiments relate to any one of the aforementioned processes, further comprising combining a compound of Formula 1:

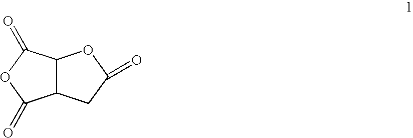

and a compound of Formula 2:

RXH 2

or a salt thereof, where X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; to give the compound of Formula 3.

Certain embodiments relate to any one of the aforementioned processes, where the compound of Formula 1 can be

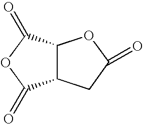

and the compound of Formula 2 can be methanol, ethanol, n-propanol, or i-propanol.

Certain embodiments relate to a process for preparing a compound of Formula 7a:

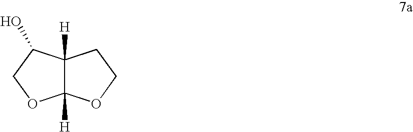

comprising (a) combining methanol, ethanol, n-propanol, or i-propanol, and a compound of Formula 1a:

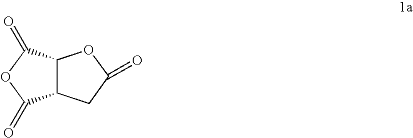

to give a compound of Formula 3a:

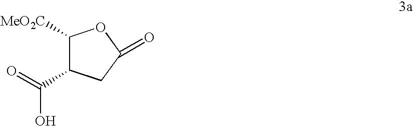

(b) combining the compound of Formula 3a, morpholine, and EDCL to give a compound of Formula 5a:

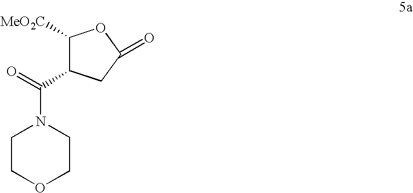

(c) combining the compound of Formula 5a with LiAlH₄ to give a compound of Formula 6a:

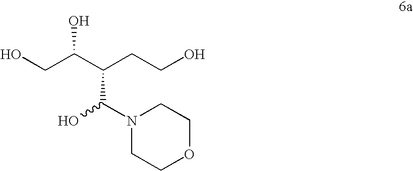

or a salt thereof; and (d) combining the compound of Formula 6a or a conjugate acid thereof, and NaHSO₄ to give the compound of Formula 7a.

Certain embodiments relate to a compound of Formula 5:

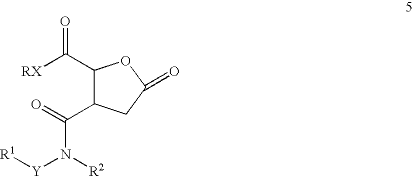

or a salt thereof, where X can be S or O; Y can be O, —N(R³)—, or a bond; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8.

Certain embodiments relate to the compound of Formula 5, where Y can be a bond; R can be alkyl; and R¹ and R² taken together with the nitrogen to which they are bonded can represent a 3-10 membered optionally substituted heterocyclic ring.

Certain embodiments relate to the compound of Formula 5, where the compound can have the absolute stereochemistry shown below:

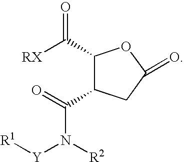

Certain embodiments relate to the compound of Formula 5, where the compound of Formula 5 can be prepared by a process comprising: (a) combining a compound of Formula 1a:

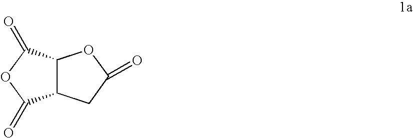

and a compound of Formula 2:

RXH 2

or a salt thereof, where X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; to give a compound of Formula 3a:

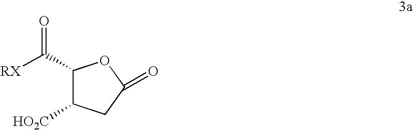

or a salt thereof, where R is as defined above; (b) combining the compound of Formula 3a with a coupling agent; and a compound of Formula 4:

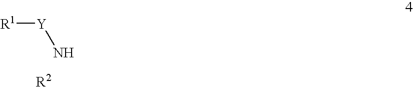

or a salt thereof, where Y can be O, —N(R³)—, or a bond; each of R¹, R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; to give the compound of Formula 5.

Certain embodiments relate to a compound of Formula 3:

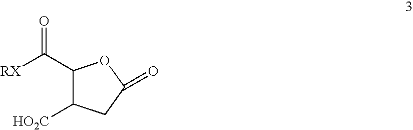

or a salt thereof, where X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; provided that when R is t-butyl, then X is not O; further provided that when R is menthyl, then X is not O; further provided that when R is benzyl, then X is not O.

Certain embodiments relate to the compound of Formula 3 where R can be alkyl and X can be O.

Certain embodiments relate to the compound of Formula 3 where the compound can have the absolute stereochemistry shown below:

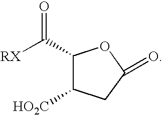

Certain embodiments relate to the compound of Formula 3 prepared by a process comprising (a) combining a compound of Formula 1a:

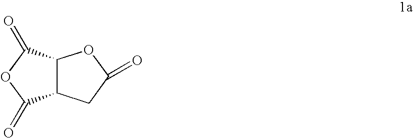

and a compound of Formula 2:

RXH 2

or a salt thereof, wherein:
X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; to give the compound of Formula 3.

Certain embodiments relate to a compound of Formula 6:

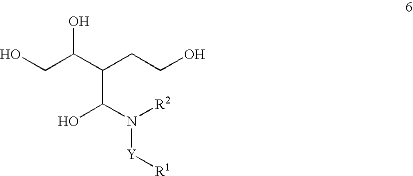

or a salt thereof, where Y can be O, —N(R³)—, or a bond; each of R¹, R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8.

Certain embodiments relate to a compound of Formula 6, where the compound can have the absolute stereochemistry shown below:

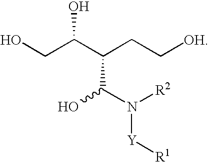

Certain embodiments relate to any of the aforementioned compounds of Formula 6, where Y can be a bond and R¹ and R² taken together with the nitrogen to which they are bonded can represent a 3-10 membered optionally substituted heterocyclic ring.

Certain embodiments relate to any one of the aforementioned compounds of Formula 6, where the compound of Formula 6 is prepared by a process comprising: (a) combining a compound of Formula 1a:

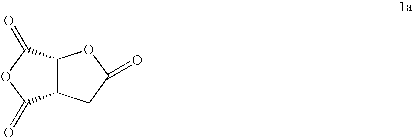

and a compound of Formula 2:

RXH 2

or a salt thereof, where X can be S or O; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; to give a compound of Formula 3a:

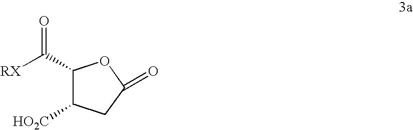

or a salt thereof, where R is as defined above; (b) combining the compound of Formula 3a with a coupling agent; and a compound of Formula 4:

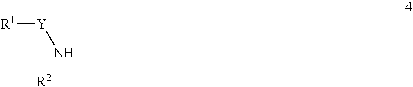

or a salt thereof, where Y can be O, —N(R³)—, or a bond; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; to give a compound of Formula 5a:

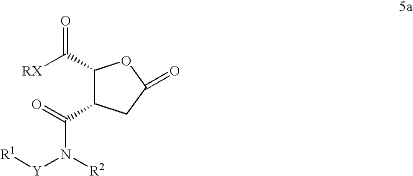

or a salt thereof, where each of R, R¹, and R², are as defined above; and (c) combining the compound of Formula 5a with a reducing agent to give the compound of Formula 6.

Certain embodiments relate a compound selected from the group consisting of:

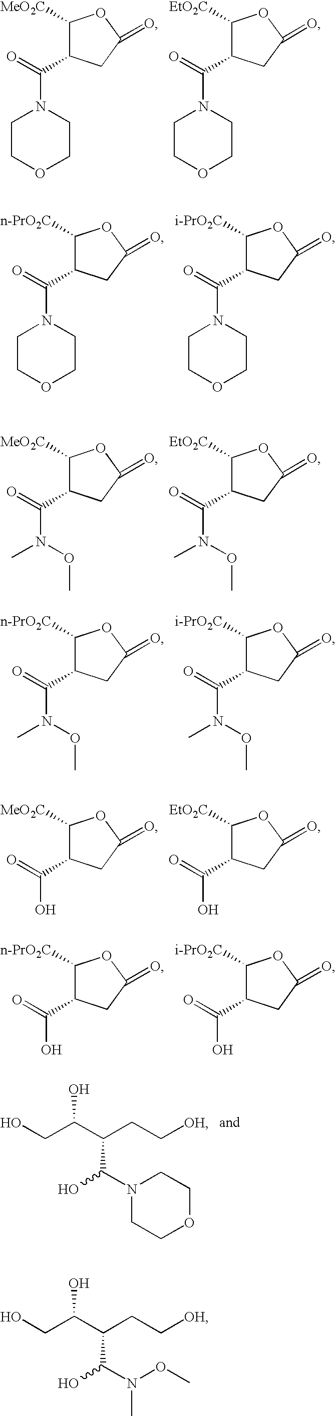

or salts thereof.

Certain embodiments relate to a process for preparing a compound of Formula 7:

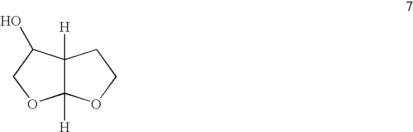

comprising (a) combining an acid and a compound of Formula 6:

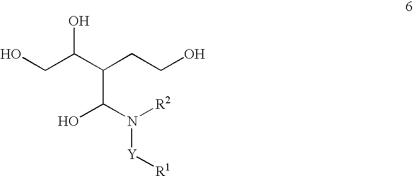

or a salt thereof, where Y can be O, —N(R³)—, or a bond; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence can be hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and m can be 0-8; to give the compound of Formula 7.

Certain embodiments relate to a process for preparing a protease inhibitor comprising (a) combining a reducing agent and a compound of Formula 5:

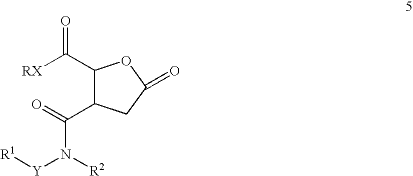

or a salt thereof, where X can be S or O; Y can be O, —N(R³)—, or a bond; R can be alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; each of R¹ R², and R³ independently for each occurrence can be hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded can represent a 3-10 membered optionally substituted heterocyclic ring; or R¹ and R³ taken together with the nitrogen to which they are bonded can represent a 3-10 membered optionally substituted heterocyclic ring; R¹⁰ independently for each occurrence can be hydrogen, alkyl, alkenyl, alkynyl, cycloalkyl, heterocycloalkyl, aryl, aralkyl, heteroaryl, or heteroaralkyl; R¹¹ independently for each occurrence can be hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together can represent a 5-8 membered optionally substituted heterocyclic ring; and m can be 0-8; to give a compound of Formula 6:

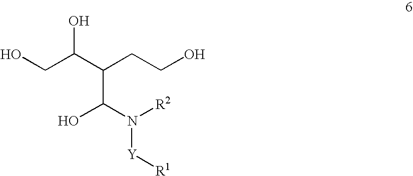

or a salt thereof, where each of Y, R¹ and R² is as defined above; (b) combining the compound of Formula 6 with an acid to give a compound of Formula 7:

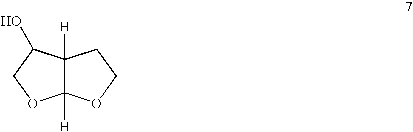

(c) combining the compound of Formula 7 with a carbamate coupling agent to give an activated carbonate; and (d) combining the activated carbonate with an amine containing protease inhibitor precursor.

Certain embodiments relate to the aforementioned process where the amine containing protease inhibitor precursor can be:
(i) a compound of Formula 13:

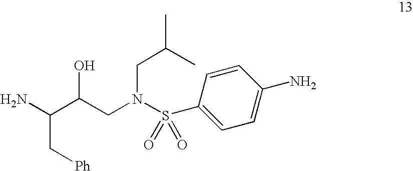

or a salt thereof; and the protease inhibitor can be a compound of Formula 13a:

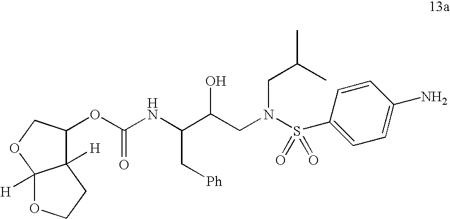

or a salt thereof;
(ii) a compound of Formula 14:

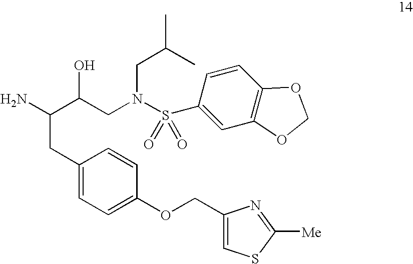

or a salt thereof; and the protease inhibitor can be a compound of Formula 14a:

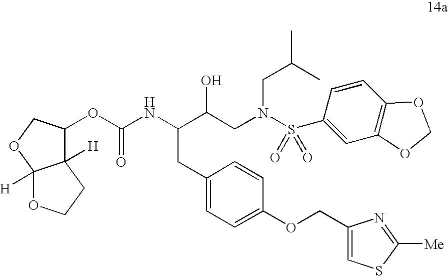

or a salt thereof;
(iii) a compound of Formula 15:

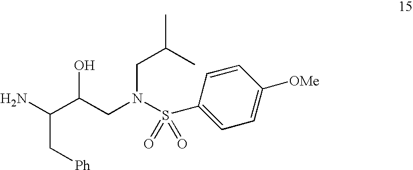

or a salt thereof; and the protease inhibitor can be a compound of Formula 15a:

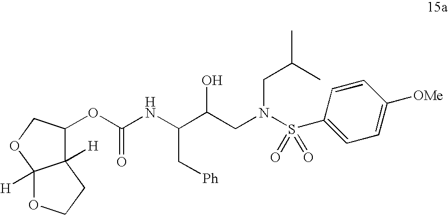

or a salt thereof;
(iv) a compound of Formula 16:

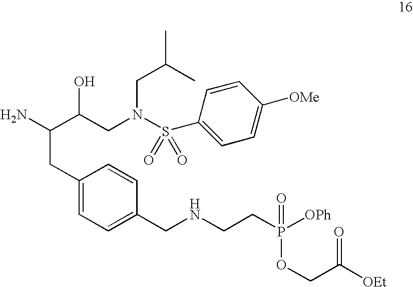

or a salt thereof; and the protease inhibitor can be a compound of Formula 16a:

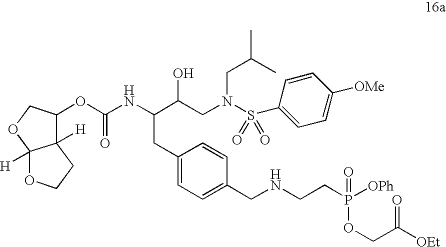

or a salt thereof;
(v) a compound of Formula 17:

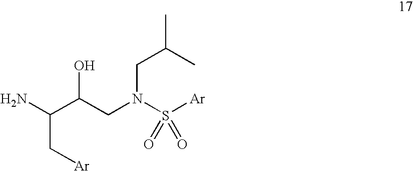

or a salt thereof; where Ar independently for each occurrence can be aryl; and the protease inhibitor can be a compound of Formula 17a:

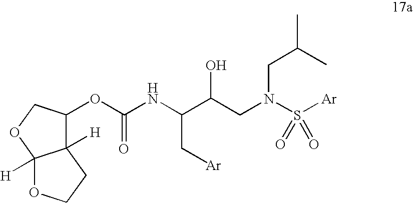

or a salt thereof; where Ar independently for each occurrence is as defined above;
(vi) a compound of Formula 18:

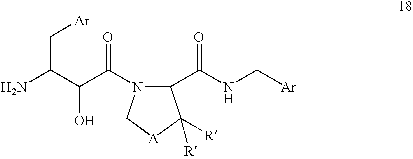

or a salt thereof, where Ar can be aryl; A can be CH₂, S, or O; and R′ independently for each occurrence can be alkyl, alkenyl, alkynyl, cycloalkyl, heterocycloalkyl, aryl, aralkyl, heteroaryl, or heteroaralkyl; and the protease inhibitor can be a compound of Formula 18a:

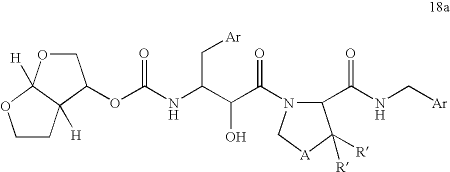

or a salt thereof; where each of Ar, A, and R′ are defined as above;
(vii) a compound of Formula 19:

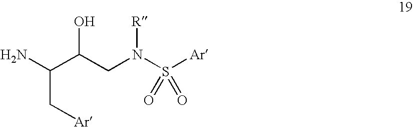

or a salt thereof, where Ar′ independently for each occurrence can be aryl optionally substituted with a water soluble oligomer and R″ can be alkyl or a water soluble oligomer, provided that at least one of Ar′ or R″ comprises a water soluble oligomer; and the protease inhibitor can be a compound of Formula 19a:

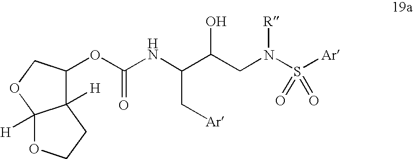

or a salt thereof; where each of Ar′ and R″ are as defined above; or
(viii) a compound of Formula 20:

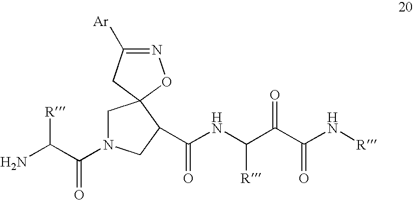

or a salt thereof, where Ar can be aryl; each of R′″ independently for each occurrence can be hydrogen, alkyl, heteroaryl, aralkyl, or heterocycloalkyl; and the protease inhibitor can be a compound of Formula 20a:

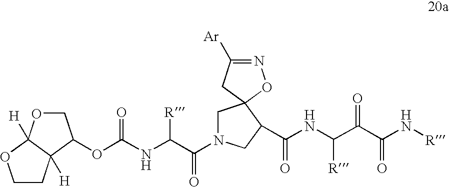

or a salt thereof; where each of Ar and R′″ are as defined above.

Certain embodiments relate to the aforementioned process, where the carbamate coupling agent can be selected from the group consisting of phosgene, trichloromethyl chloroformate, bis(trichloromethyl) carbonate, bis(4-nitrophenyl)carbonate, bis(pentafluorophenyl) carbonate, N,N′-disuccinimidyl carbonate, 4-nitrophenyl chloroformate, 2,2′-dipyridyl carbonate, and N,N′-carbonyldiimidazole (CDI).

Certain embodiments relate to any one of the aforementioned processes where R can be alkyl; X can be O; and each of R¹ and R² independently for each occurrence can be alkyl, cycloalkyl, aryl, or —[C(R¹⁰)₂]ₘ—R¹¹, wherein independently for each occurrence R¹⁰ can be hydrogen or alkyl, R¹¹ is alkoxy; or R¹ and R² taken together with the nitrogen to which they are bonded can represent a 5-8 membered optionally substituted heterocyclic ring.

Certain embodiments relate to a protease inhibitor prepared by any of the aforementioned processes.

Other features, objects, and advantages of the compounds and methods described herein will be apparent from the description and drawings, and from the claims.

### DETAILED DESCRIPTION

### Definitions

The term “heteroatom” refers to an atom of any element other than carbon or hydrogen. Illustrative heteroatoms include boron, nitrogen, oxygen, phosphorus, sulfur and selenium.

The term “alkyl” includes saturated aliphatic groups, including straight-chain alkyl groups and branched-chain alkyl groups. In certain embodiments, a straight chain or branched chain alkyl has about 30 or fewer carbon atoms in its backbone (e.g., C₁-C₃₀ for straight chain, C₁-C₃₀ for branched chain), and alternatively, about 20 or fewer. In certain instances, alkyl groups can be optionally substituted.

The term “cycloalkyl” include saturated, cycloalkyl groups, alkyl substituted cycloalkyl groups, and cycloalkyl substituted alkyl groups. Cycloalkyls include monocyclic and polycylic rings. Cycloalkyls can have from about 3 to about 15 carbon atoms in their ring structure, and alternatively about 5, 6, 7, or 10 carbons in the ring structure. In certain instances, cycloalkyl groups can be optionally substituted.

The term “aryl” includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms “heterocyclyl”, “heteroaryl”, or “heterocyclic group” include 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described herein, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “optionally substituted” refers to any chemical group, such as alkyl, cycloalkyl aryl, and the like, wherein one or more hydrogens may be replaced with a a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like; or has the formula —[(CR₅₀R₅₁)ₙ]R₅₂, wherein each of R₅₀ and R₅₁ independently for each occurrence is hydrogen, alkyl, aralkyl, cycloalkyl, or aryl; R₅₂ is hydrogen, amino, acylamino, nitro, sulfhydryl, imino, amido, phosphonate, phosphinate, carbonyl, carboxyl, silyl, ether, alkylthio, sulfonyl, sulfonamido, ketone, aldehyde, ester; and n is an integer selected from 1, 2, 3, 4, 5, 6, 7, 8, 9, and 10.

The term “salt” includes any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary ions include, but are not limited to chloride, bromide, iodide, nitrate, sulfate, bisulfate, sulfite, phosphate, acid phosphate, chlorate, perchorate, hypochlorite, iodate, periodate, hypoiodite, carbonate, bicarbonate, isonicotinate, acetate, trichloroacetate, trifluroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluormethylbenzenesulfonate, hydroxide, earth metals, such as aluminium (e.g., aluminates) and boron (e.g., borates and tetraborates), alkali metals, such as lithium, sodium, potassium, and cesium, alkaline earth metals, such as beryllium, magnesium, calcium, strontium, and barium, silver, zinc, ammounium salts.

The definition of each expression, e.g., alkyl, m, n, and the like, when it occurs more than once in any structure, is intended to be independent of its definition elsewhere in the same structure.

As used herein, chemical structures which contain one or more stereocenters depicted with wedged shaped bonds, i.e., [symbol] are meant to indicate absolute stereochemistry of the stereocenter(s) present in the chemical structure. As used herein, chemical structures which contain one or more stereocenters depicted with non-wedge shaped bonds, i.e., [symbol] or [symbol] are meant to indicate relative stereochemistry of the stereocenter(s) present in the chemical structure. Unless otherwise indicated to the contrary, chemical structures, which include one or more stereocenters, illustrated herein without indicating absolute or relative stereochemistry encompass all possible steroisomeric forms of the compound (e.g., diastereomers, enantiomers, cis/trans isomers, etc) and mixtures thereof.

Provided herein are efficient methods for the preparation of hexahydro-furo[2,3-b]furan-3-ol 7. The methods employed can be modified to access different stereoisomers, enantiomers, and diastereomers of hexahydro-furo[2,3-b]furan-3-ol from readily available precursors. Compound 7 can be prepared in 4 steps from isocitric acid lactone anhydride 1 (Scheme 1).

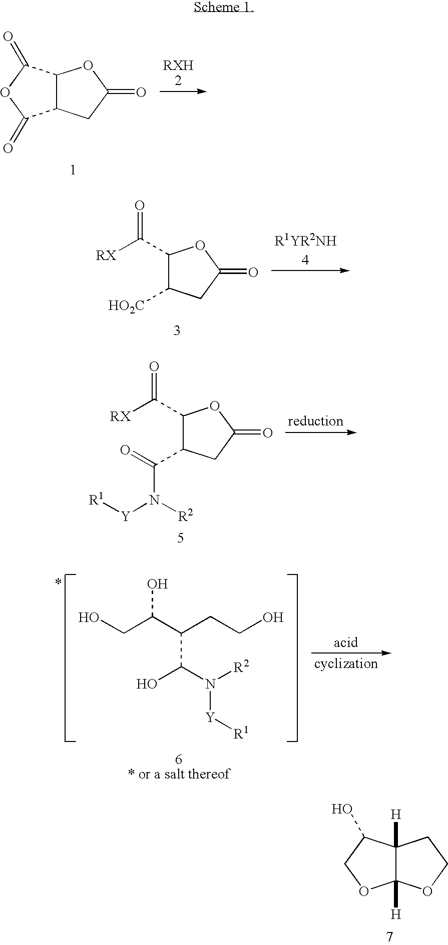

Compound 1 can be prepared according to the procedure described in DE226473, from isocitric acid (e.g., (2R,3S), (2S, 3R), racemic isocitric acid), which is readily produced on large scale by fermentation (DE2065207, JP35014494, JP50155683). The anhydride lactone 1 can be reacted with a nucleophile, e.g., an alcohol, to yield carboxylic acid 3. Carboxylic acid 3 can be coupled with, e.g., an amine to yield compound 5, which can then be reacted with a reducing agent, such as LiAlH₄, and subjected to acid catalyzed cyclization to yield the desired compound 7. Optically enriched hexahydro-furo[2,3-b]furan-3-ol is readily available by starting from optically enriched lactone anhydride 1, or by separating the desired enantiomer at any step in the synthesis starting from racemic compound 1.

Anhydride 1 when reacted with nucleophile 2, such as an alcohol or thiol, under suitable conditions, undergoes anhydride ring opening in a regioselective fashion to afford compound 3. In certain instances, the nucleophile is an alcohol, thiol, or salts thereof. Examples of nucleophiles useful for reaction with the anhydride include, but are not limited to methanol, ethanol, n-propanol, sec-propanol, n-butanol, sec-butanol, tert-butanol, benzyl alcohol, methylthiol, n-propanthiol, sec-propanthiol, n-butanthiol, sec-butanthiol, tert-butanthiol, and benzylthiol.

In certain instances, the nucleophile 2 is a compound of formula RXH, where X is S or O; R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹, where R¹⁰ independently for each occurrence is hydrogen, alkyl, alkenyl, alkynyl, cycloalkyl, heterocycloalkyl, aryl, aralkyl, heteroaryl, or heteroaralkyl; and R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and m is 0-8. In the examples provided below, RXH is methanol.

In certain instances, a salt of nucleophile 2 can be used for the anhydride ring opening. Such salts include alkali metal, alkaline earth metal, and ammonium salts of the nucleophile. The anhydride ring opening can be accomplished using a number of well known procedures. For example, an alcohol or thiol can be reacted with compound 1 to afford compound 3. In certain instances, the alcohol or thiol can serve as the solvent for the reaction. Other solvents may be used in this reaction, such as tetrahydrofuran, tetrahydropyran, diethyl ether, methyl tert-butyl ether, 1,4-dioxane, 1,2-dimethoxyethane, ethylacetate, acetonitrile, dichloromethane, chloroform, carbon tetrachloride, 1,2-dichloroethane, acetone, dimethylformamide, dimethylsulfoxide, hexamethylphosphoramide, alcohols, water, and mixtures thereof.

The reaction can be run at a temperature from about —10° C. to about 120° C., from about −10° C. to about 100° C., from about −10° C. to about 80° C., from about −10° C. to about 60° C., form about —10° C. to about 40° C., from about 0° C. to about 40° C., or from about 20° C. to about 30° C. In certain instances, the reaction can be run from about −30° C. to about 0° C., from about −10° C. to about 30° C., from about 30° C. to about 70° C., from about 70° C. to about 120° C. In certain instances, more reactive nucleophiles, such salts of alcohols and tniols, can be run at lower temperatures. In the examples below, methanol is used as the nucleophile and the reaction is run at room temperature.

An organic or inorganic base can optionally be used in the reaction of the nucleophile 2 (e.g., alcohol or thiol) with compound 1. Inorganic bases, such as alkali and alkali earth, oxides, hydroxides, carbonates, bicarbonates, and hydrides; and ammonium hydroxide can be used in the anhydride ring opening. Organic bases useful in the anhydride ring opening include but are not limited to tertiary amines, such as triethylamine N,N-diisopropylethyl amine (Huinig's base), N-methyl morpholine, and 1,4-diazabicyclo[2.2.2]octane (DABCO), 1,8-diazabicyclo[5.4.0]undec-7-ene (DBU), 1,5-diazabicyclo[4.3.0]non-5-ene (DBN), pyridine, imidazole, and alkali and alkaline earth metal alkoxides, such as sodium tert-butoxide.

Lewis acids can also be utilized to catalyze the addition of the nucleophile 2 to the anhydride. Lewis acids useful in the reaction include, but are not limited to rare earth salts, such as scandium, ytterbium, and lanthanum, magnesium, zinc salts, manganese, cobalt, copper, and silver salts.

The crude carboxylic acid 3 can either be used directly in the next reaction, without purification, or purified prior to the acylation reaction. The crude lactone can be purified by any number of techniques, including liquid-liquid extraction, solid-liquid extraction, chromatography, distillation and crystallization.

The carboxylic acid 3 can be reacted directly or indirectly with nucleophile 4 under conditions suitable to form compound 5. Indirect methods for forming compound 5 include first preparing an activated carboxyl intermediate, which is then reacted with nucleophile 4. Examples of reagents useful for preparing activated carboxyl intermediates include, halogenating agents, such as SOCl₂, SO₂Cl₂, PCl₃, PBr₃, POCl₃, POBr₃, oxalyl chloride, dichlorotriphenylphosphorane, and N,N-dimethylchloromethylenammonium chloride, 1,1-carbonyldiimidazole (CDI), and reagents which generate mixed anhydrides, such as pivaloyl chloride and isobutyl chloroformate (IBCF). The resulting activated carboxyl containing compound can then be reacted with an amine as described herein. A base can optionally be used in the acylation reaction. In certain instances, the amine to be coupled acts as the base.

In certain instances, the carboxylic acid 3 is activated in situ and the resulting activated carboxyl containing compound is reacted with the nucleophile 4 to afford compound 5. In situ generation of the activated carboxyl compounds can provide synthetic efficiencies and lower material costs. Reagents useful for generating the activated carboxyl include, but are not limited to carboiimides, such as 1-tert-butyl-3-ethylcarbodiimide, N,N′-di-tert-butylcarbodiimide, N,N′-dicyclohexylcarbodiimide, N,N′-diisopropylcarbodiimide, N-(3-dimethylaminopropyl)-N′-ethylcarbodiimide, 1-[3-(dimethylamino)propyl]-3-ethylcarbodiimide methiodide, and 1,3-di-p-tolylcarbodiimide, phosphonium reagents, such as (7-azabenzotriazol-1-yloxy)tripyrrolidinophosphonium hexafluorophosphate, (benzotriazol-1-yloxy)tripyrrolidinophosphonium hexafluorophosphate, (benzotriazol-1-yloxy)tris(dimethylamino)phosphonium hexafluorophosphate, bromotripyrrolidinophosphonium hexafluorophosphate, bromotris(dimethylamino)phosphonium hexafluorophosphate, and chlorotripyrrolidinophosphonium hexafluorophosphate, uronium reagents such as o-(7-azabenzotriazol-1-yl)-N,N,N′,N′-tetramethyluronium hexafluorophosphate, o-benzotriazol-1-yl-N,N,N′,N′-bis(pentamethylene)uronium hexafluorophosphate, and o-(benzotriazol-1-yl)-N,N,N′,N′-bis(tetramethylene)uronium hexafluorophosphate, formamidinium such as chloro-N,N,N′,N′-bis(tetramethylene)formamidinium tetrafluoroborate and chloro-N,N,N′,N′-tetramethylformamidinium hexafluorophosphate, imidazolidinium reagents such as 2-chloro-1,3-dimethylimidazolidinium chloride and 2-fluoro-1,3-dimethylimidazolidinium hexafluorophosphate, 4-(4,6-dimethoxy-1,3,5-triazin-2-yl)-4-methylmorpholinium chloride (DMTMM), and 2-chloro-4,6-dimethoxy-1,3,5-triazine/N-methylmorpholine (CDMT/NMM). Methods employing in situ formation of the activated carboxyl containing compound can further include an organic or inorganic base and additional coupling agents, such as N-hydroxybenzotriazole. Suitable organic bases include tertiary amines, such as Hunig's base, triethylamine, N-methyl morpholine, piperidine and pyridine. Inorganic bases, including alkali and alkali earth carbonates, bicarbonates, hydroxide, and alkoxides can be used in the coupling reaction.

Various nucleophiles can be used in the acylation reaction. The nucleophile 4 can be a primary or secondary amine, a hydrazine, or alkoxyamine. Examples of suitable amines include, but are not limited to pyrrolidine, piperidine, and morpholine, or can be represented by the formula R¹YNR², where Y can be a bond (i.e., a single bond) between N and R¹ or oxygen; each of R¹ and R² independently for each occurrence can be C1-C8-alkyl, C5-C8-cycloalkyl, optionally substituted phenyl or benzyl; or R¹ is a C1-C8-alkyl, C5-C8-cycloalkyl, optionally substituted phenyl or benzyl, and R² is a C1-C8-alkyloxy, C5-C8-cycloalkyloxy; or R¹ and R² together with the N atom form a five- to eight-membered optionally substituted ring. In certain instances, R¹ and R² are identical radicals, e.g., each C1-C4-alkyl, such as methyl, ethyl, n- or i-propyl or n-, i- or t-butyl; or R¹ is a C1-C4-alkyl, such as methyl, ethyl, n-propyl, i-propyl or n- or i-butyl, and R² is a C1-C4-alkyloxy, such as methoxy, ethoxy, n- or i-propyloxy, n- or i- or t-butyloxy; or Y is oxygen, R¹ is methyl and R² is methoxy.

In certain instances, the nucleophile 4 used in the acylation reaction is represented by R¹YNR², where Y is a bond (i.e., a single bond between R¹ and N) between N and R¹, oxygen, or —N(R³)—; each of R¹, R², and R³ independently for each occurrence is hydrogen, alkyl, alkenyl, alkynyl, cycloalkyl, heterocycloalkyl, aryl, aralkyl, heteroaryl, heteroaralkyl, haloalkyl (i.e., alkyl radicals having one or more halogens), or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring; R¹⁰ independently for each occurrence is hydrogen, alkyl, alkenyl, alkynyl, cycloalkyl, heterocycloalkyl, aryl, aralkyl, heteroaryl, or heteroaralkyl; R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together form a 5-8 membered optionally substituted heterocyclic ring; and m is 0-8.

In the examples below, EDCL and HOBT are used to couple morpholine to carboxylic acid 3 in acetonitrile. Other solvents useful in the reaction include, but are not limited to propionitrile, dichloromethane, dichloroethane, chloroform, tetrahydrofuran, tetrahydropyran, 1,4-dioxane, 1,2-dimethoxyethane, dimethylformamide, dimethylsulfoxide, methylacetate, ethylacetate, n-propyl acetate, isopropyl acetate, n-butyl acetate, isobutyl acetate, and hexamethylphosphoramide. The acylation reaction can be run at any temperature from about −40 to about 100° C. (e.g., about −40° C. to about 0° C., about −20° C. to about 20° C., about 20° C. to about 60° C., or about 60° C. to about 100° C.). In certain instances the acylation reaction is run at about room temperature or about 0° C.

Crude compound 5 can be purified prior to reduction or can be reduced without purification. Any number of methods can be used to purify compound 5, including liquid-liquid extraction, solid-liquid extraction, chromatography, and crystallization.

Compound 5 can then be reacted with a reducing agent. In certain instances, the reducing agent is any reagent capable of reducing the lactone and ester or thioester functional groups to primary alcohols and the amide to an N, O-animal or aldehyde. Reducing agents useful for reducing compound 5 include but are not limited to zinc, N(R⁴)₄, alkali (e.g., Li, Na, and K), and alkali earth salts (e.g., Ca) of ⊖BH₄, ⊖HBR⁴₃, ⊖H₂BR⁴₂, ⊖H₃BR⁴, ⊖HB(OR⁴)₃, ⊖H₂B(OR⁴)₂, and ⊖H₃BOR⁴, boranes, such as HBR⁴₂, H₂BR⁴, and BH₃, including borane amine complexes, and aluminum reducing agents, such as ⊖AlH₄, ⊖HAlR⁴₃, ⊖H₂AlR⁴₂, ⊖H₃AlR⁴, ⊖HAl(OR⁴)₃, ⊖H₂Al(OR⁴)₂, and ⊖H₃AlOR⁴, and organo-aluminum reagents such as HAlR⁴₂, H₂AlR⁴, and AlH₃, including aluminum amine complexes, where R⁴ independently for each occurrence is alkyl or aralkyl. In reaction sequences illustrating the reduction step, i.e., the transformation of compound 5 into compound 6, (i.e., Scheme 1) “M” can be a cation, e.g., zinc(I), ⊕N(R⁴)₄, alkali(I) (e.g., Li, Na, and K), and alkali earth(II) salts (e.g., Ca and Mg), where R⁴ is as defined above.

Suitable solvents for the reduction include diethyl ether, diisopropyl ether, tetrahydrofuran, 1,4-dioxane, tert-butyl methyl ether, or alcohols, such as methanol, ethanol, n-propanol, iso-propanol, n-butanol, s-butanol, tert-butanol, and mixtures thereof. The reduction can be run at temperatures of from about −100° C. to about 80° C. (e.g., about −100° C. to about −40° C., about −40° C. to about 20° C., about 20° C. to about 40° C., about 40° C. to about 100° C., or about −80° C. to about 0° C.). Protic solvents, such as water and alcohols, can be used to increase the reactivity of the reducing agent employed.

In certain instances, reaction of a reducing agent with compound 5 yields a salt of compound 6. Salts of compound 6 include alkali, alkali earth, boron (e.g., borate ester, boronic ester, borinic ester), aluminum (e.g., aluminates, aluminum alkoxides, and organo-aluminum alkoxides), and ammonium salts, mixtures thereof, and/or polymeric complexes thereof. When the phrase “salts thereof” is used in connection with compounds of Formula 6 it is meant to include the crude product(s) of the reduction reaction of compounds of Formula 5 with a reducing agent, e.g., boron and aluminum reducing agents described herein.

Compound 6 and salts thereof can be subjected directly to acid catalyzed cyclization to afford the desired product 7. This can be accomplished by adding an acid directly to the reaction mixture after reduction of compound 5 has run to completion. In certain instances, the conjugate acid of compound 6 can be isolated as the aldehyde, hydrate, or cyclic hemi-acetal, and mixtures thereof illustrated in Scheme 2. These isolated compounds or mixtures thereof can then be subjected to the acid catalyzed cyclization conditions.

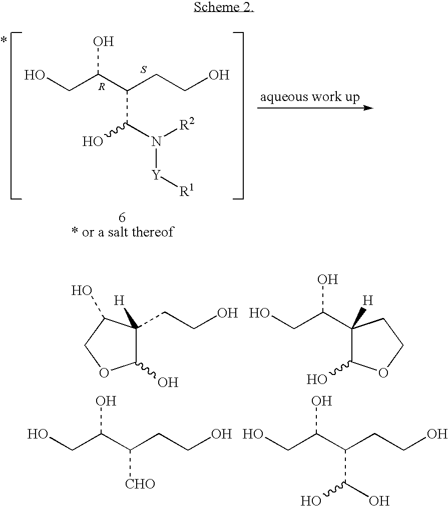

Suitable acids for the cyclization reaction include Brønsted and Lewis acids. Brønsted acids useful in the cyclization reaction include, but are not limited to inorganic acids, such as hydrochloric acid, hydrobromic acid, hydroiodic acid, nitric acid, sulfuric acid, metal hydrogen sulfate, sulfurous acid, metal hydrogensulfite, phosphoric acid, metal dihydrogen phosphate, metal hydrogen phosphate, phosphonic acid, metal hydrogen phosphate, pyrophosphoric acid, metal trihydrogen pyrophosphate, metal dihydrogen pyrophosphate, and metal hydrogen pyrophosphate, and organic acids, such as citric, fumaric, maleic, malic, ascorbic, succinic, tartaric, benzoic, acetic, trifluoroacetic acid, trichloracetic acid, methanesulfonic, trifluormethanesulfonic acid, ethanesulfonic, benzenesulfonic, p-toluenesulfonic acid, p-trifluoromethylbenzenesulfonic acid, camphorsulfonic acid, naphthalene-1,5-disulfonic acid, ethan-1,2-disulfonic acid, cyclamic acid, thiocyanic acid, naphthalene-2-sulfonic acid, and oxalic acid.

Lewis acids can also be employed in the acid catalyzed cyclization reaction. Such Lewis acids include, but are not limited to TMSOTf, AlCl₃, Al(OR⁴)₃, BF₃, BCl₃, SbF₅, SnCl₄, TiCl₄, Ti(OR⁴)₄, where R⁴ independently for each occurrence is as defined above.

The acid catalyzed cyclizition reaction can be conducted in any solvent, including but not limited to diethyl ether, diisopropyl ether, tetrahydrofuran, tetrahydropyran 1,4-dioxane, tert-butyl methyl ether, dichloromethane, dichloroethane, chloroform, carbontetrachloride, acetonitrile, 1,4-dioxane, 1,2-dimethoxyethane, dimethylformamide, dimethylsulfoxide, ethylacetate, hexamethylphosphoramide, water and mixtures thereof.

In the examples below, NaHSO₄ is used as the acid for the cyclization reaction in a mixture of water and tetrahydrofuran.

Enantiomerically and/or diastereomerically enriched hexahydro-furo[2,3-b]furan-3-ol can be prepared using optically active starting materials. Chirality present in the starting material can be preserved through the synthetic process. For example, (3R,3aS,6aR)-hexahydro-furo[2,3-b]furan-3-ol can be prepared from (2R,3S) isocitric acid as illustrated in Scheme 3.

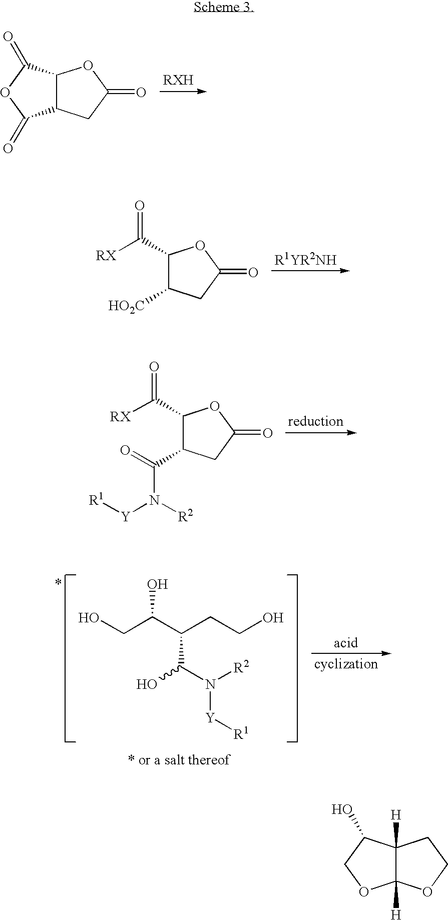

Optically enriched hexahydro-furo[2,3-b]furan-3-ol can also be prepared from racemic starting material and separating optical isomers at any step in the synthesis using any method known to those of ordinary skill in the art, e.g., separating optical isomers using chiral chromatography (e.g., HPLC or SFC using columns with chiral stationary phase) or by forming diasteromers with an optically enriched compound, e.g., optically active amines can be used to make diasteromeric salts (e.g., with compound 3), diasteremeric amides (e.g., of compound 5) and separated using traditional purification techniques, or by enzymatic resolution of a racemic mixture (e.g. compound 7 or its esters) using the appropriate esterase enzyme.

Some or all of the steps described herein can be conducted in the same reaction vessel, e.g., as a “one pot method” or in different reaction vessels. Reactions conducted in the same reaction vessel can be run in the same or different solvents. Solvent transfers can be used when changing solvents between synthetic steps, e.g., at the end of a particular reaction the solvent is removed (e.g, by distillation), and another solvent can be added. For example, the anhydride ring opening reaction can be conducted in methanol, after the reaction is complete, the methanol can be removed, reagents and solvents for the acylation step (i.e., the reaction of compound 3 with nucleophile 4 to give compound 5) can be added to the reaction vessel, and the acylation reaction can be carried our in the same vessel. In another example, after compound 3 is subjected to the reduction reaction, an acid can be added directly to the reduction reaction mixture in the same reaction vessel to perform the acid catalyzed cyclization.

Using the methods described herein hexahydro-furo[2,3-b]furan-3-ol can be prepared efficiently and in high yield from readily available isocitric acid lactone anhydride 1. The synthetic routes described herein can provide the final product in at least 40%, at least, 50%, at least 60%, at least 70%, at least 80%, at least 90% or at least 95% overall yield from isocitric acid lactone anhydride 1.

Certain compounds are useful synthetic intermediates in the processes described herein. These compounds include compounds of Formula 3, 5, and 6. For example, compounds of Formula 3 are useful in the acylation reaction with nucleophile 4. Compounds of Formula 3 can be represented by:

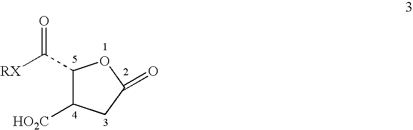

or a salt thereof, where X can be S or O; R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; R¹⁰ independently for each occurrence is hydrogen, alkyl, alkenyl, alkynyl, cycloalkyl, heterocycloalkyl, aryl, aralkyl, heteroaryl, or heteroaralkyl; R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and m is 0-8; provided that when R is t-butyl, then X is not O; further provided that when R is menthyl, then X is not O; further provided that when R is benzyl, X is not O. In certain instances, the compound of Formula 3 can have a cis relationship between groups attached at carbons labelled 4 and 5 above. In certain instances, the compound of Formula 3 can have the absolute stereochemistry depicted below:

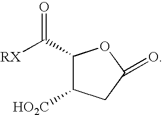

In certain instances, R can be alkyl, cycloalkyl, haloalkyl, aryl, or aralkyl; and X can be O or S; or R can be alkyl and X can be O.

In certain instances, the compound of Formula 3 can be:

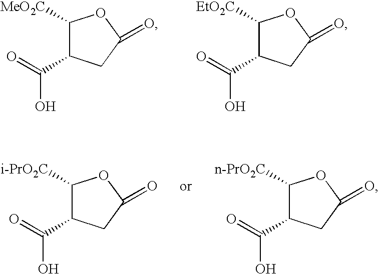

or salts thereof.

In certain instances, the compound of Formula 3 is prepared according to a method as described herein.

Compounds of Formula 5 are useful in the methods as described herein. Compounds of Formula 5 can be represented by:

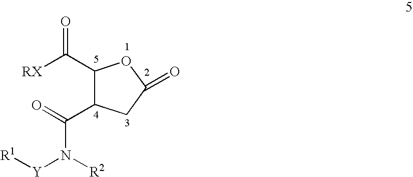

or a salt thereof where: X is S or O; Y is O, —N(R³)—, or a bond; R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; each of R¹, R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring; R¹⁰ independently for each occurrence is hydrogen, alkyl, alkenyl, alkynyl, cycloalkyl, heterocycloalkyl, aryl, aralkyl, heteroaryl, or heteroaralkyl; R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹²), —OC(O)OR¹², —CO₂R¹², —OC(O)R¹², —C(O)N(OR¹²)(R¹³), or —SO₂N(R¹²)(R¹³), —N(R¹²)S(O)₂OR¹³; each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and m is 0-8. In certain instances, the compound of Formula 5 can have a cis relationship between groups attached at carbons labelled 4 and 5 above. In certain instances, the compound of Formula 5 can have the absolute stereochemistry depicted below:

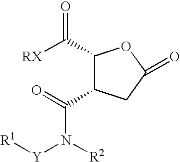

In certain instances, R is alkyl, Y is a bond, and R¹ and R² taken together with the nitrogen to which they are bonded represent a 3-10 membered optionally substituted heterocyclic ring.

In certain instances, the compound of Formula 5 can be:

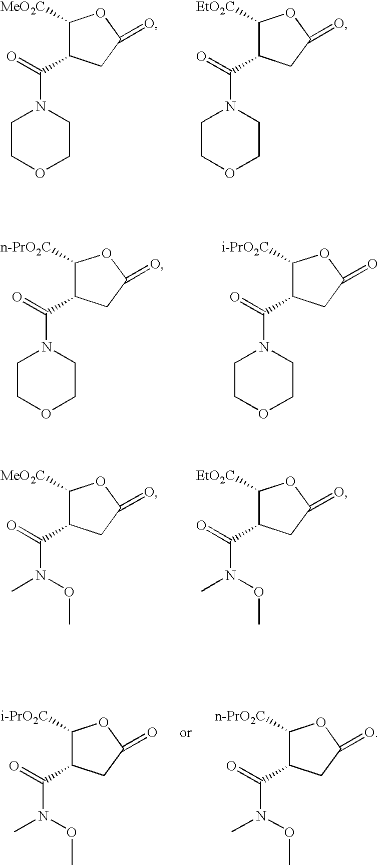

In certain instances, the compound of Formula 5 is prepared according to a method as described herein.

Compounds of Formula 6 are useful in the methods as described herein. Compounds of Formula 6 can be represented by:

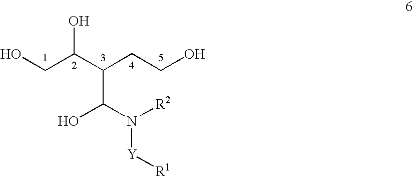

or a salt thereof, where: Y is O, —N(R³)—, or a bond; R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; each of R¹, R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring; R¹⁰ independently for each occurrence is hydrogen, alkyl, alkenyl, alkynyl, cycloalkyl, heterocycloalkyl, aryl, aralkyl, heteroaryl, or heteroaralkyl; R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂O R¹³; and each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and m is 0-8. In certain instances, the compound of Formula 6 can have a syn relationship (e.g., 2S, 3R and 2R, 3S as labelled above) between groups attached at carbons labelled 2 and 3 above. In certain instances, the compound of Formula 6 can have the absolute stereochemistry depicted below:

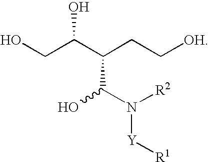

In certain instances, Y is a bond and R¹ and R² taken together with the nitrogen to which they are bonded represent a 3-10 membered optionally substituted heterocyclic ring, e.g., pyrrolidine, piperidine, and morpholine.

In certain instances, the compound of Formula 6 can be:

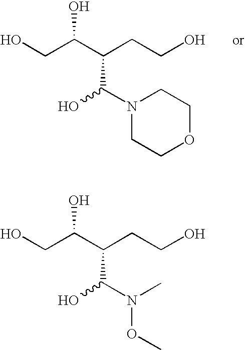

or a salt thereof.

In certain instances, the compound of Formula 6 is prepared according to a method as described herein.

A number of HIV protease inhibitors contain the hexahydro-furo[2,3-b]furan-3-ol (Compound 7) moiety. Examples of such protease inhibitors include darunavir, brecanevir, UIC-94003, and GS-9005 (shown below), which incorporate (3R,3aS,6aR) hexahydro-furo[2,3-b]furan-3-ol.

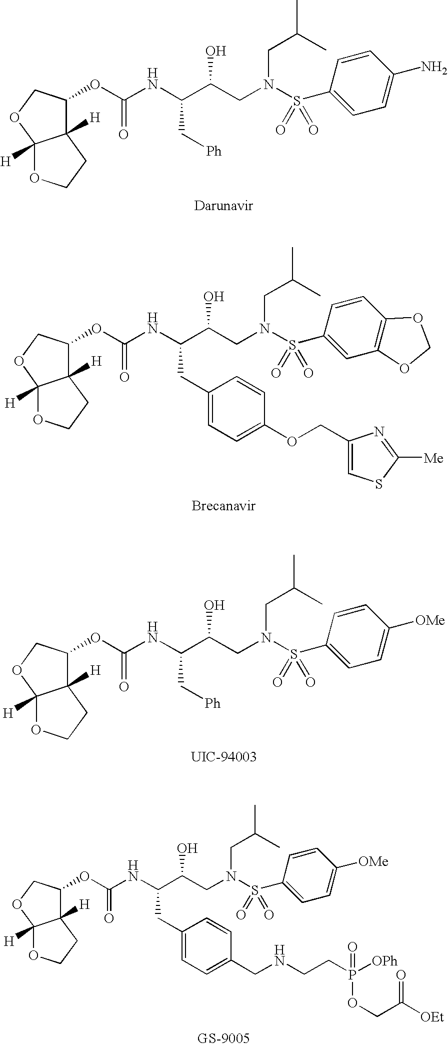

Other HIV protease inhibitors incorporate hexahydro-furo[2,3-b]furan-3-ol, such as the compounds described in published Japanese patent application number JP20050478474, published PCT application W02008112289 (herein incorporated by reference). These compounds include those represented by structures 18a and 19a illustrated below.

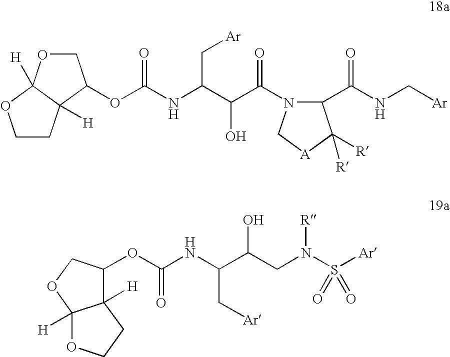

Compounds represented by structure 18a include compounds where Ar is an aryl group; A is CH₂, S, or O, and R′ independently for each occurrence is alkyl, alkenyl, alkynyl, cycloalkyl, heterocycloalkyl, aryl, aralkyl, heteroaryl, or heteroaralkyl. Compounds of structure 19a include compounds where R″ is an alkyl or a water soluble oligomer, e.g., a polyalkylene oxide, a polyolefinic alcohol, a polyhydroxyalkyl methacrylamide a polymethacrylate, and a poly-N-acryloylmorpholine; and Ar′ independently for each occurrence is aryl optionally substituted with a water soluble oligomer; provided that at least one of Ar′ or R″ comprises a water soluble oligomer.

The hexahydro-furo[2,3-b]furan-3-ol moiety is also incorporated in certain HCV protease inhibitors. Published PCT applications WO2007025307 and WO2008106139 (herein incorporated by reference) describe the use of HCV protease inhibitors that incorporate the hexahydro-furo[2,3-b]furan-3-ol moiety. These compounds included those represented by structure 20a illustrated below.

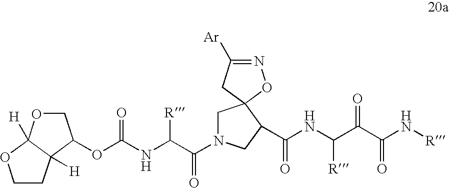

where R′″independently for each occurrence is hydrogen, alkyl, heteroaryl, aralkyl, or heterocycloalkyl; and Ar is an aryl group.

The methods and intermediates described herein can be used to prepare the protease inhibitors described above and other protease inhibitors that contain the hexahydro-furo[2,3-b]furan-3-ol moiety.

In the compounds shown above, the hexahydro-furo[2,3-b]furan-3-ol moiety is attached via a carbamate linker to an amine. Any method known to one of ordinary skill in the art can be employed for coupling hexahydro-furo[2,3-b]furan-3-ol using a carbamate linker. Such methods include the steps of reacting hexahydro-furo[2,3-b]furan-3-ol or a salt thereof with a carbamate coupling agent to give an activated hexahydro-furo[2,3-b]furan-3-yl carbonate; and combining the activated carbonate with an amine containing protease inhibitor precursor. In other instances, the protease inhibitor is prepare by first reacting an amine containing protease inhibitor precursor with a carbamate coupling agent to give an activated protease inhibitor precursor carbonate, and combining hexahydro-furo[2,3-b]furan-3-ol or a salt thereof, to give a coupled carbonate (as illustrated below).

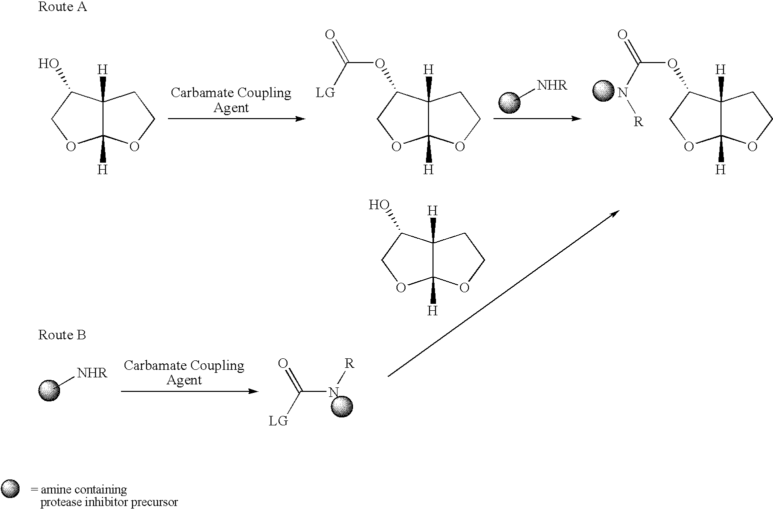

The carbamate coupling reaction can be the final step in the synthesis of the protease inhibitor, in which case the product of the reaction is the desired protease inhibitor, or can be done at an earlier step in the synthetic sequence.

Any method known for coupling an amine to an alcohol via a carbamate linker can be employed at the carbamate coupling step in the preparation of the protease inhibitor. Coupling agents useful in the coupling reaction include, but are not limited to phosgene, trichloromethyl chloroformate, bis(trichloromethyl) carbonate, bis(4-nitrophenyl)carbonate, bis(pentafluorophenyl) carbonate, N,N′-disuccinimidyl carbonate, 4-nitrophenyl chloroformate, 2,2′-dipyridyl carbonate, and N,N′-carbonyldiimidazole (CDI).

In certain instances, a base is added to the coupling reaction. Such bases include organic and inorganic bases including, but not limited to tertiary amines, such as triethylamine, diisopropylethylamine, and N-methyl morpholine, 1,8-diazabicyclo[5.4.0]undec-7-ene (DBU), 1,5-diazabicyclo[4.3.0]non-5-ene (DBN), imidazole, and pyridine; and inorganic bases, including NaH, alkali and alkali earth carbonates, bicarbonates, and hydroxides.

The coupling reaction can be performed in any solvent, including, but not limited to acetonitrile, propionitrile, dichloromethane, dichloroethane, chloroform, tetrahydrofuran, tetrahydropyran, 1,4-dioxane, 1,2-dimethoxyethane, dimethylformamide, dimethylsulfoxide, methylacetate, ethylacetate, n-propyl acetate, isopropyl acetate, n-butyl acetate, isobutyl acetate, hexamethylphosphoramide, and mixtures thereof. The carbamate coupling reaction can be conducted at temperatures ranging from about −40° C. to about 100° C. (e.g., about −40° C. to about −0° C., about 0° C. to about 30° C., about 30° C. to about 60° C., or about 60° C. to about 100° C.). In certain instances the coupling reaction is conducted at about 0° C. or about room temperature.

Protease inhibitors can be prepared by reacting an activated hexahydro-furo[2,3-b]furan-3-yl carbonate with the following amine containing protease inhibitor precursors:

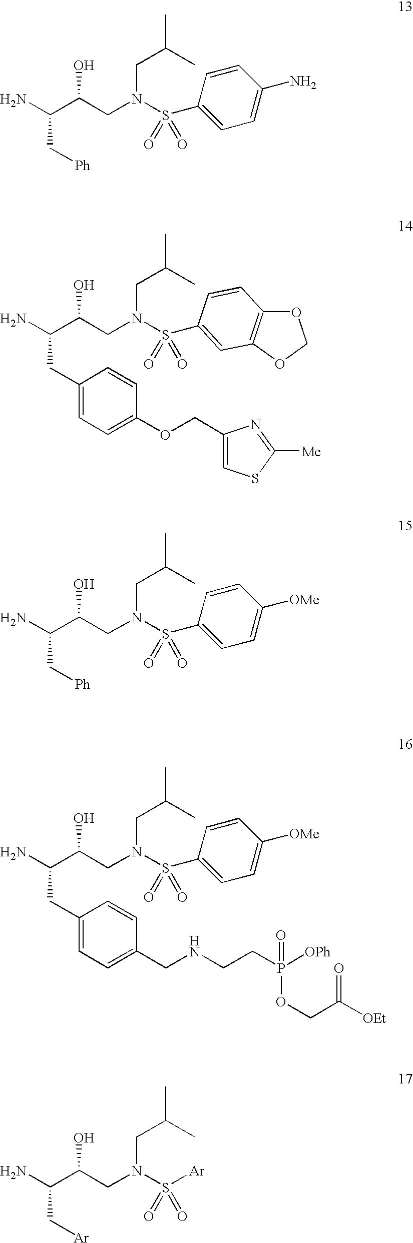

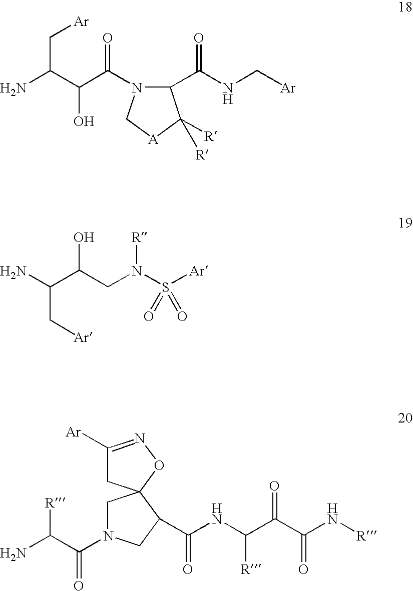

or a salt thereof and/or a suitably protected derivative thereof, where Ar, Ar′, R′, R″, and R′″ are as defined above.

### EXAMPLES

The following examples serve to illustrate the process of the present invention without limiting the scope thereof.

### Example 1

### Synthesis of Hexahydrofuro[2,3-b]furan-3-ol

### Step A.

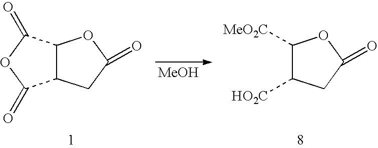

A suspension of racemic cis-isocitric acid lactone anhydride (2.30 g, 14.8 mmol) in methanol (15 mL) was stirred for 20 h at room temperature. The solvent was removed under reduced pressure to give the product (2.78 g, 100%) as an oil. ¹H NMR (CDCl₃, 300 MHz): δ 2.79 (dd, 1H, J=9.2 and 17.7 Hz), 3.09 (dd, 1H, J=9.5 and 17.7 Hz), 3.77 (dd, 1H, J=9.2 and 17.5 Hz), 3.80 (s, 3H), 5.13 (d, 1H, J=8.5 Hz). ¹³C NMR (CDCl₃, 75 MHz): δ 30.16, 43.28, 53.16, 76.14, 168.15, 173.05, 173.49.

### Step B.

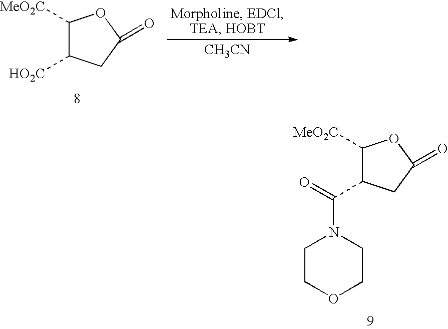

To a stirring solution of compound 8 (2.63 g, 14 mmol) in acetonitrile (20 mL) was added HOBT (2.45 g, 16 mmol) and EDCl (2.88 g, 15 mmol). After the mixture was stirred at room temperature for 20 min, morpholine (1.30 mL, 15 mmol) and Et₃N (2.10 mL, 15 mmol) were added. The mixture was stirred overnight at room temperature. After removal of acetonitrile under reduced pressure, the residue was partitioned between 4 M HCl (10 mL) and CH₂Cl₂ (50 mL). The organic layer was separated and the aqueous layer was extracted with dichloromethane (2×15 ml). The combined organic layers were dried (MgSO₄) and concentrated under reduced pressure. The residue was purified by chromatography (SiO₂, EtOAc-hexane: 1: 1) to give the product 9 (3.31 g, 92%) as white crystals. ¹H NMR (CDCl₃, 300 MHz): δ 2.61 (dd, 1H, J=9.7 and 17.5 Hz), 3.28 (dd, 1H, J=10.2 and 17.7 Hz), 3.57 (m, 4H), 3.70 (m, 4H), 3.78 (s, 3H), 3.95 (dd, 1H, J=8.8 and 17.5 Hz), 5.03 (d, 1H, J=8.6 Hz). ¹³C NMR (CDCl₃, 75 MHz): δ 30.48, 41.45, 42.79, 46.11, 52.96, 66.30, 66.54, 76.00, 166.05, 168.00, 174.32.

### Step C.

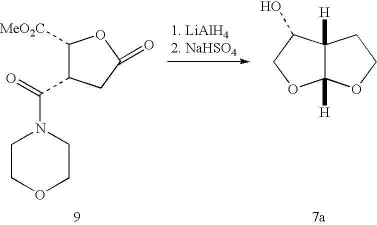

To a solution of compound 9 (515 mg, 2 mmol) in THF (10 mL) was added dropwise 1 M LiAlH₄ solution in THF at −78° C. After stirring for 1 h at −78° C., the cooling bath was removed, and the mixture was stirred for another 1 h. The mixture was cooled to −10° C. and 50% NaHSO₄ aqueous solution was added dropwise. The mixture was stirred overnight at room temperature and dried (MgSO₄). After filtration, the filtrate was concentrated under reduced pressure. The residue was purified by chromatography (SiO₂, EtOAc-hexane: 4:1) to give product 7a (198 mg, 76%) as pale yellow oil. ¹H NMR (CDCl₃, 300 MHz): δ 1.89(m, 1H), 2.05 (m, 1H), 2.32 (m, 1H), 2.87 (m, 1H), 3.64(dd, 1H, J=7.1 and 8.6 Hz), 3.92 (m, 1H), 3.99 (m, 2H), 4.46 (m, 1H), 5.70 (d, 1H, J=5.0 Hz). ¹³C NMR (CDCl₃, 75 MHz): δ 24.93, 46.54, 69.92, 70.63, 73.02, 109.53.

A number of embodiments of a method for preparing hexahydrofuro[2,3-b]furan-3-ol and intermediates useful therein have been described. Nevertheless, it will be understood that various modifications may be made without departing from the spirit and scope of the disclosure. Accordingly, other embodiments are within the scope of the following claims.

What is claimed is:

1. A process for preparing a compound of Formula 7: 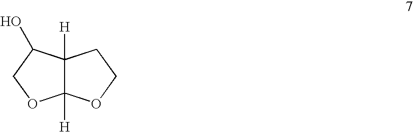
comprising:
(a) combining a reducing agent and a compound of Formula 5: 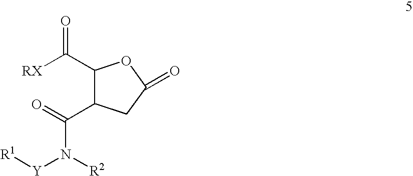
or a salt thereof, wherein:
X is S or O;
Y is O, —N(R³)—, or a bond;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
R¹⁰ independently for each occurrence is hydrogen, alkyl, alkenyl, alkynyl, cycloalkyl, heterocycloalkyl, aryl, aralkyl, heteroaryl, or heteroaralkyl; or
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give a compound of Formula 6: 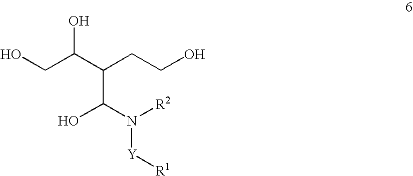
or a salt thereof, wherein: each of Y, R¹ and R² is as defined above; and combining said compound of Formula 6 with an acid to give said compound of Formula 7.

2. The process of claim 1, wherein said reducing agent is MBH₄, MHB(R³)₃, MH₂B(R⁴)₂, MH₃BR⁴, MHB(OR⁴)₃, MH₂B(OR⁴)₂, MH₃BOR⁴, MAlH₄, MHAl(OR⁴)₃, MH₂Al(OR⁴)₂, MH₃Al(OR⁴), HB(R⁴)₂, H₂BR⁴, BH₃, H₂Al(R⁴)₂, H₂AlR⁴, or H₃Al;
M is Li, Na, K, R³₄N, ½Zn or ½Ca; and
R⁴ is alkyl or aralkyl.

3. The process of claim 1, wherein said acid is hydrochloric acid, hydrobromic acid sulfuric acid, phosphoric acid, nitric acid, metal hydrogen sulfate, metal dihydrogen phosphorate, trifluoroacetic acid, trichloroacetic acid, citric acid, oxalic acid, tartaric acid, oxalic acid, formic, methanesulfonic acid, trifluoromethanesulfonic acid, benzenesulfonic acid, p-toluenesulfonic acid, 10-camphorsulfonic acid, 1,5-napthalene disulfonic acid, or 1,2-ethane disulfonic acid.

4. The process of claim 1, wherein said compound of Formula 7 is

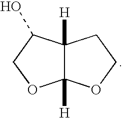

5. The process of claim 1, wherein R is alkyl.

6. The process of claim 1, wherein Y is a bond; and each of R¹ and R² independently for each occurrence is alkyl, cycloalkyl, aryl, or —[C(R¹⁰)₂]ₘ—R¹¹, wherein independently for each occurrence R¹⁰ is hydrogen or alkyl, R¹¹ is alkoxy; or R¹ and R² taken together with the nitrogen to which they are bonded represent a 5-8 membered optionally substituted heterocyclic ring.

7. The process of claim 1, wherein Y is a bond; R is alkyl; and each of R¹ and R² independently for each occurrence is alkyl, cycloalkyl, aryl, or —[C(R¹⁰)₂]ₘ—R¹¹, wherein independently for each occurrence R¹⁰ is hydrogen or alkyl, R¹¹ is alkoxy; or R¹ and R² taken together with the nitrogen to which they are bonded represent a 5-8 membered optionally substituted heterocyclic ring.

8. The process of claim 1, wherein Y is a bond; R is methyl, ethyl, n-propyl, or i-propyl; X is O; and R¹ and R² taken together with the nitrogen to which they are bonded form a heterocyclic ring selected from piperidine, pyrrolidine, or morpholine.

9. The process of claim 1, further comprising:
combining a compound of Formula 3: 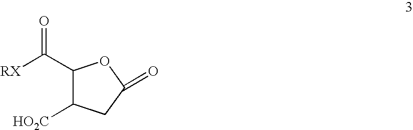
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
a coupling agent; and
a compound of Formula 4: 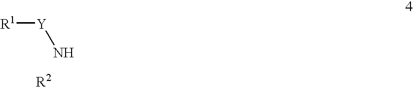
or a salt thereof, wherein
Y is O, —N(R³)—, or a bond;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
to give said compound of Formula 5.

10. The process of claim 9, wherein said coupling agent is dicyclohexylcarbodiimide (DCC), diisopropylcarbodiimide (DIC), N,N′-carbonyldiimidazole (CDI), 3-(diethoxyphosphoryloxy)-1,2,3-benzotriazin-4(3H)-one (DEPBT), 1-ethyl-3-(3-dimethyllaminopropyl)carbodiimide hydrochloride (EDCL). 2-(7-aza-1H-benzotriazole-1-yl)-1,1,3,3-tetramethyluronium hexafluorophosphate (HATU). 2-(1H-benzotriazole-1-yl)-1,1,3,3-tetramethyluronium hexafluorophosphate (HABTU), 2-(6-chloro-1H-benzotriazole-1-yl)-1,1,3,3-tetramethylaminium hexafluorophosphate (HCTU), benzotriazole-1-yl-oxy-tris-pyrrolidino-phosphonium hexafluorophosphate (PyBOP). bromo-tris-pyrrolidino-phosphonium hexafluorophosphate (PyBrOP). O-(7-azabenzotriazole-1-yl)-N,N,N′,N′-tetramethyluronium tetrafluoroborate (TATU), 2-(1H-benzotriazole-1-yl)-1,1,3,3-tetramethyluronium tetrafluoroborate (TBTU). N,N,N′,N′-tetramethyl-O-(3,4-dihydro-4-oxo-1,2,3-benzotriazin-3-yl)uronium tetrafluoroborate(TBTU), O-(N-succinimidyl)-1,1,3,3-tetramethyluronium tetrafluoroborate (TSTU), 4-(4,6-dimethoxy-1,3,5-triazin-2-yl)-4-methylmorpholinium chloride (DMTMM), 2-chloro-4,6-dimethoxy-1,3,5-triazine/N-methylmorpholine (CDMT/NMM), oxalyl chloride, SOCl₂, SO₂Cl₂, POCl₃, PCl₃, PCl₅, PBr₃, PBr₅POBr₃, pivaloyl chloride, or pivaloyl anhydride.

11. The process of claim 9, wherein said coupling agent is CDI, DMTMM, or EDCL and said compound of Formula 4 is morpholine.

12. The process of claim 10, wherein R is methyl, ethyl, n-propyl, or i-propyl; X is O; and said compound of Formula 4 is morpholine.

13. The process of claim 9, further comprising:
combining a compound of Formula 1: 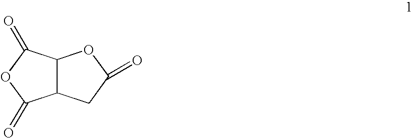
and a compound of Formula 2:
RXH 2
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR², —CO₂R², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give said compound of Formula 4.

14. The process of claim 13, wherein said compound of Formula I is 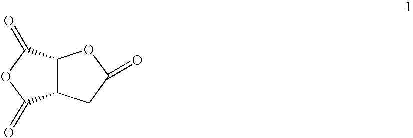
and said compound of Formula 2 is methanol, ethanol, n-propanol, or i-propanol.

15. A process for preparing a compound of Formula 7a: 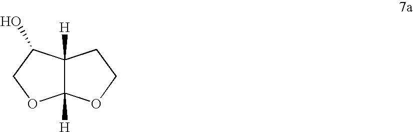
comprising:
(a) combining methanol, ethanol, n-propanol, or i-propanol, and a compound of Formula 1a: 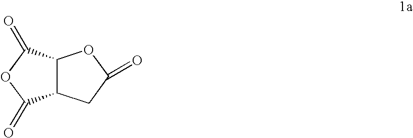
to give a compound of Formula 3a: 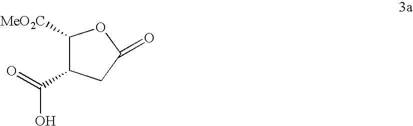
(b) combining said compound of Formula 3a, morpholine, and EDCL to give a compound of Formula 5a: 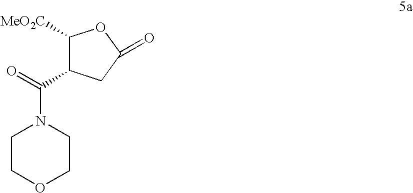
(c) combining said compound of Formula 5a with LiAlH₄ to give a compound of Formula 6a: 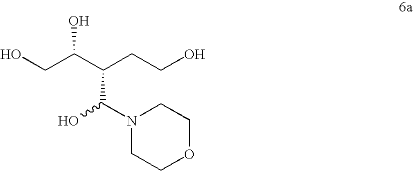
or a salt thereof, and
(d) combining said compound of Formula 6a or a conjugate acid thereof, and NaHSO₄ to give said compound of Formula 7a.

16. A compound of Formula 5: 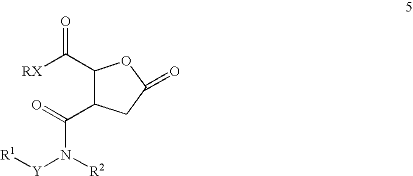
or a salt thereof, wherein:
X is S or O;
Y is O, —N(R³)—, or a bond;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R²)(R³), —N(R²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8.

17. The compound of claim 16, wherein Y is a bond; R is alkyl; and R¹ and R² taken together with the nitrogen to which they are bonded represent a 3-10 membered optionally substituted heterocyclic ring.

18. The compound of claim 16, wherein said compound has the absolute stereochemistry shown below:

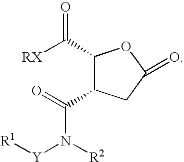

19. The compound of claim 16, wherein said compound of Formula 5 is prepared by a process comprising:
(a) combining a compound of Formula 1a: 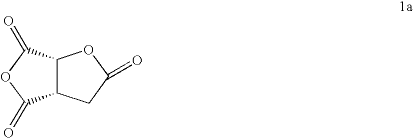
and a compound of Formula 2:
RXH 2
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give a compound of Formula 3a: 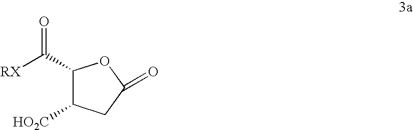
or a salt thereof, wherein: R is as defined above;
(b) combining said compound of Formula 3a with a coupling agent; and
a compound of Formula 4: 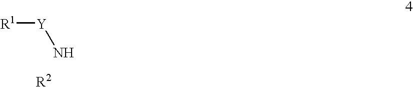
or a salt thereof, wherein
Y is O, —N(R³)—, or a bond;
each of R¹, R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
to give said compound of Formula 5.

20. A compound of Formula 3a: 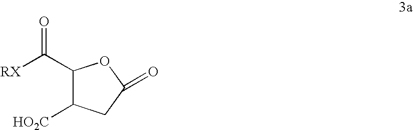
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R12)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
provided that when R is 1-butyl, then X is not O;
further provided that when R is menthyl, then X is not O
further provided that when R is benzyl, then X is not O.

21. The compound of claim 20, wherein R is alkyl and X is O.

22. The compound of claim 20, wherein said compound has the absolute stereochemistry shown below:

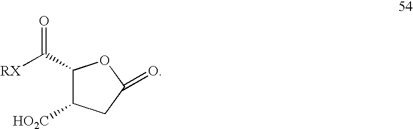

23. The compound of claim 20, wherein said compound of Formula 3a is prepared by a process comprising:
(a) combining a compound of Formula 1a: 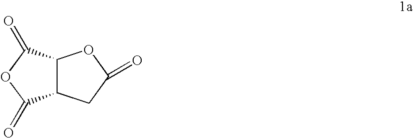
and a compound of Formula 2:
RXH 2
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, −OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give said compound of Formula 3a.

24. A compound of Formula 6a: 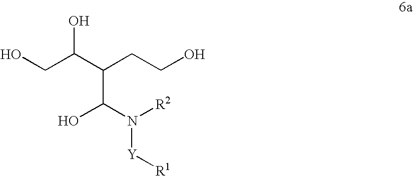
or a salt thereof, wherein:
Y is O, —N(R³)—, or a bond;
each of R¹, R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8.

25. The compound of claim 24, wherein said compound has the absolute stereochemistry shown below:

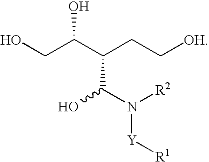

26. The compound of claim 24, wherein Y is a bond and R¹ and R² taken together with the nitrogen to which they are bonded represent a 3-10 membered optionally substituted heterocyclic ring.

27. The compound of claim 24, wherein said compound of Formula 6a is prepared by a process comprising:
(a) combining a compound of Formula 1a: 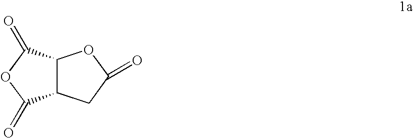
and a compound of Formula 2:
RXH 2
or a salt thereof, wherein:
X is S or O;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give a compound of Formula 3a: 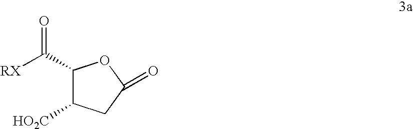
or a salt thereof, wherein: R is as defined above;
(b) combining said compound of Formula 3a with a coupling agent; and
a compound of Formula 4: 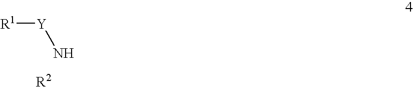
or a salt thereof, wherein
Y is O, —N(R³)—, or a bond;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
to give a compound of Formula 5a: 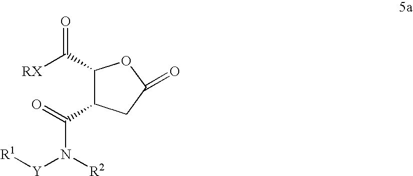
or a salt thereof, wherein each of R, R¹, and R², are as defined above; and
(c) combining said compound of Formula 5a with a reducing agent to give said compound of Formula 6a.

28. A compound selected from the group consisting of: 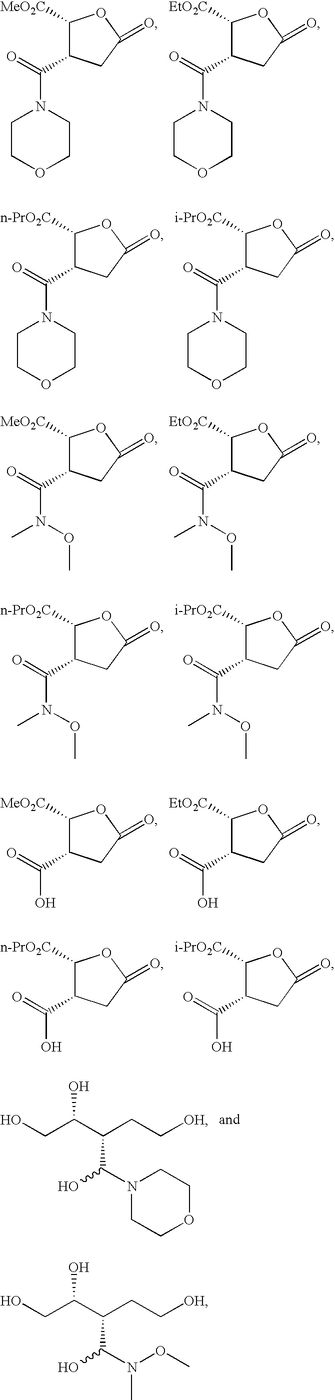
or salts thereof.

29. A process for preparing a compound of Formula 7: 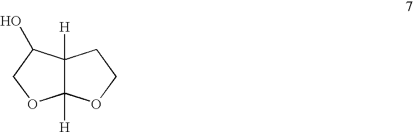
comprising:
(a) combining an acid and a compound of Formula 3: 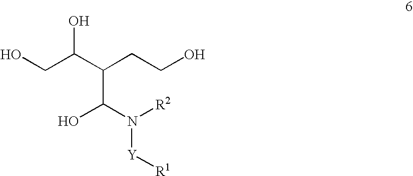
or a salt thereof, wherein:
Y is O, —N(R³)—, or a bond;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8; to give said compound of Formula 7.

30. A process for preparing a protease inhibitor comprising:
(a) combining a reducing agent and a compound of Formula 5a: 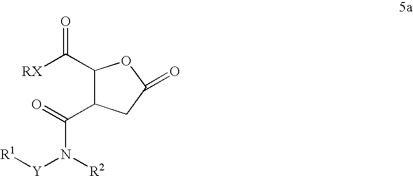
or a salt thereof, wherein:
X is S or O;
Y is O, —N(R³)—, or a bond;
R is alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹;
each of R¹ R², and R³ independently for each occurrence is hydrogen, alkyl, alkenyl, alkynyl, cycloalkyl, heterocycloalkyl, aryl, aralkyl, heteroaryl, heteroaralkyl, haloalkyl, or —[C(R¹⁰)₂]ₘ—R¹¹; or R¹ and R² taken together with the nitrogen to which they are bonded represent a 3-10 membered optionally substituted heterocyclic ring; or R¹ and R³ taken together with the nitrogen to which they are bonded represent a 3-10 membered optionally substituted heterocyclic ring;
R¹⁰ independently for each occurrence is hydrogen, alkyl, alkenyl, alkynyl, cycloalkyl, heterocycloalkyl, aryl, aralkyl, heteroaryl, or heteroaralkyl;
R¹¹ independently for each occurrence is hydrogen, alkyl, alkenyl, alkynyl, cycloalkyl, heterocycloalkyl, aryl, aralkyl, heteroaryl, heteroaralkyl, halide, nitrile, nitro, —OR¹², —N(R¹²)COR¹³, —N(R¹²)C(O)OR¹³, —N(R¹²)SO₂(R¹³), —CON(R¹²)(R¹³), —OC(O)N(R¹²)(R¹³), —OC(O)OR¹², —CO₂R¹², —OC(O)R¹², —C(O)N(OR¹²)(R¹³), or —SO₂N(R¹²)(R¹³), —N(R¹²)S(O)₂OR¹³;
each of R¹² and R¹³ independently for each occurrence is hydrogen, alkyl, alkenyl, alkynyl, cycloalkyl, heterocycloalkyl, aryl, aralkyl, heteroaryl, heteroaralkyl; or R¹² and R¹³ taken together represent a 5-8 membered optionally substituted heterocyclic ring; and
m is 0-8;
to give a compound of Formula 6a: 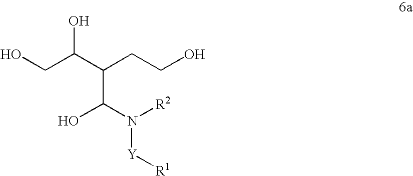
or a salt thereof, wherein: each of Y, R¹ and R² is as defined above;
(b) combining said compound of Formula 6a with an acid to give a compound of Formula 7a: 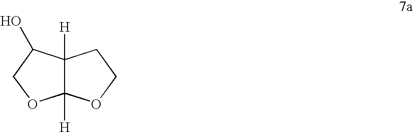
(c) combining-said compound of Formula 7a with a carbamate coupling agent to give an activated carbonate; and
(d) combining said activated carbonate with an amine containing protease inhibitor precursor.

31. The process of claim 30, wherein said amine containing protease inhibitor precursor is:
(i) a compound of Formula 13: 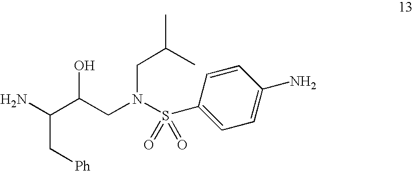
or a salt thereof; and said protease inhibitor is a compound of Formula 13a: 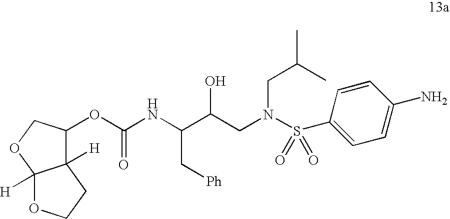
or a salt thereof;
(ii) a compound of Formula 14: 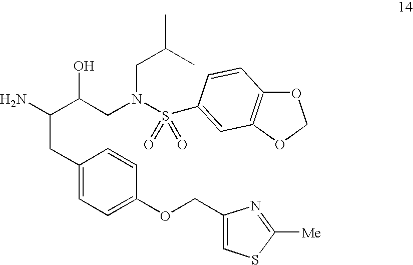
or a salt thereof; and said protease inhibitor is a compound of Formula 14a: 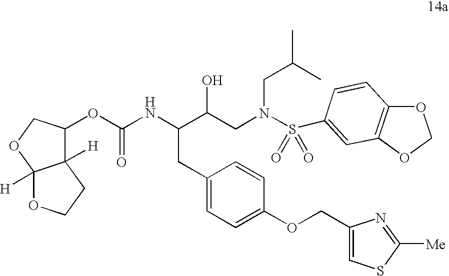
or a salt thereof;
(iii) a compound of Formula 15: 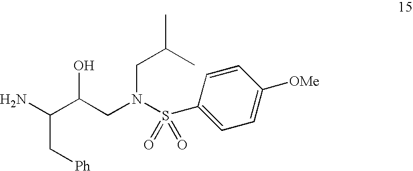
or a salt thereof; and said protease inhibitor is a compound of Formula 15a: 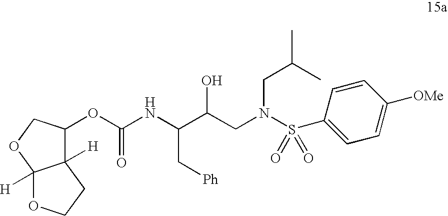
or a salt thereof;
(iv) a compound of Formula 16: 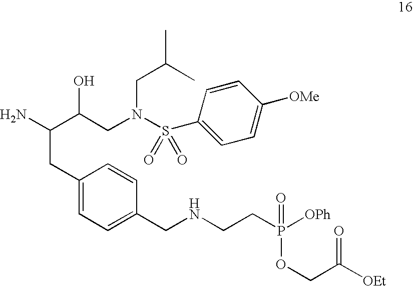
or a salt thereof; and said protease inhibitor is a compound of Formula 16a: 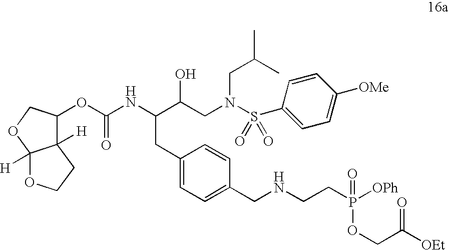
or a salt thereof;
(v) a compound of Formula 17: 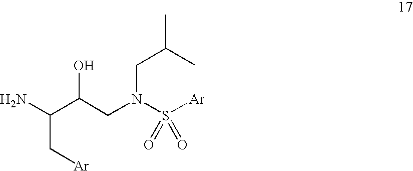
or a salt thereof; wherein Ar independently for each occurrence is aryl; and said protease inhibitor is a compound of Formula 17a: 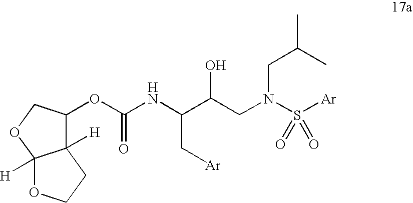
or a salt thereof; wherein Ar independently for each occurrence is as defined above;
(vi) a compound of Formula 18: 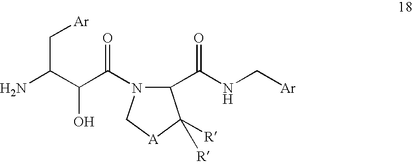
or a salt thereof, wherein Ar is aryl; A is CH₂, S, or O; and R′ independently for each occurrence is alkyl, alkenyl, alkynyl, cycloalkyl, heterocycloalkyl, aryl, aralkyl, heteroaryl, or heteroaralkyl; and said protease inhibitor is a compound of Formula 18a: 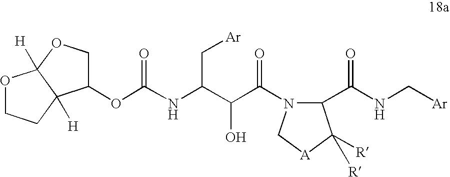
or a salt thereof; wherein each of Ar, A, and R′ are defined as above;
(vii) a compound of Formula 19: 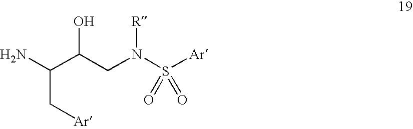
or a salt thereof, wherein Ar′ independently for each occurrence is aryl optionally substituted with a water soluble oligomer and R″ is an alkyl or a water soluble oligomer, provided that at least one of Ar′ or R″ comprises a water soluble oligomer; and said protease inhibitor is a compound of Formula 19a: 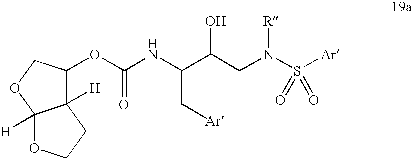
or a salt thereof; wherein each of Ar′ and R″ are as defined above; or
(viii) a compound of Formula 20: 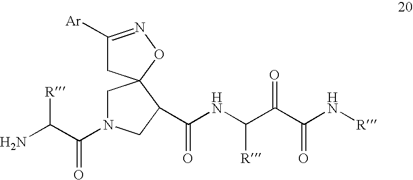
or a salt thereof, wherein Ar is aryl; each of R′″ independently for each occurrence is hydrogen, alkyl, heteroaryl, aralkyl, or heterocycloalkyl; and said protease inhibitor is a compound of Formula 20a: 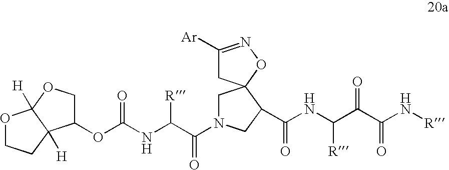
or a salt thereof; wherein each of Ar and R′″ are as defined above.

32. The process of claim 30, wherein said carbamate coupling agent is selected from the group consisting of phosgene, trichloromethyl chloroformate, bis(trichloromethyl) carbonate, bis(4-nitrophenyl) carbonate, bis(pentafluorophenyl) carbonate, N,N′-disuccinimidyl carbonate, 4-nitrophenyl chloroformate, 2,2′-dipyridyl carbonate, and N,N′-carbonyldiimidazole (CDI).

33. The process of claim 30, wherein R is alkyl; X is O; and each of R¹ and R² independently for each occurrence is alkyl, cycloalkyl, aryl, or —[C(R¹⁰)₂]ₘ—R¹¹, wherein independently for each occurrence R¹⁰ is hydrogen or alkyl, R¹¹ is alkoxy; or R¹ and R² taken together with the nitrogen to which they are bonded represent a 5-8 membered optionally substituted heterocyclic ring.

34. A protease inhibitor prepared by the process of claim 30.
